(19) United States
(12) Patent Application Publication
(10) Pub. No.: US 2017/0265782 A1
(43) Pub. Date: Sep. 21, 2017

(54) MOTION TRIGGERED VITAL SIGN MEASUREMENT

(71) Applicant: KONINKLIJKE PHILIPS N.V., EINDHOVEN (NL)

(72) Inventor: THOMAS VOLLMER, AACHEN (DE)

(21) Appl. No.: 15/310,277

(22) PCT Filed: Apr. 23, 2015
(86) PCT No.: PCT/IB2015/052964

§ 371 Date: Nov. 10, 2016

Related U.S. Application Data
(60) Provisional application No. 61/991,640, filed on May 12, 2014.

(51) Int. Cl. A61B 5/11; A61B 8/08; A61B 5/021; A61B 5/022; A61B 5/00

(52) U.S. Cl. CPC A61B 5/1116; A61B 5/1126; A61B 5/022; A61B 5/7275; A61B 5/746; A61B 5/02125; A61B 5/1112; A61B 8/08; A61B 2562/0219; A61B 5/024

(57) ABSTRACT

A medical monitoring device (12) includes a motion detection device (20, 50) and a vital sign monitoring device (26, 52) communicatively connected to the motion detection device. The motion detection device (20, 50) detects whole body motion of a person. The vital sign monitoring device (26, 52) monitors, either continuously or intermittently, at least one vital sign for a predetermined time interval in response to the detected whole body motion. The vital sign is evaluated, e.g. for orthostatic intolerance, and in response to an affirmative evaluation an alarm signal is issued.

The following relates generally to medical monitoring. It finds particular application in conjunction with vital sign monitoring and motion detection, and will be described with particular reference thereto. However, it will be understood that it also finds application in other usage scenarios and is not necessarily limited to the aforementioned application.

People suffer from hemodynamic problems such as orthostatic hypotension, which can result in a patient falling and injuring themselves. For example, a person moving from a sitting position to a standing position can suffer from syncope or fainting, which can occur from 0-45 minutes after standing according to current medical guidelines.

Current methods to diagnosis orthostatic hypotension typically include a tilt table test in a hospital setting. The person is placed on a horizontal table surface, which rotates to a vertical or near vertical orientation, and the person is monitored for symptoms of syncope.

Patients are monitored non-invasively for vital signs, such as ECG, blood pressure, pulse, etc. Devices which monitor vital signs typically monitor either continuously or when manually initiated. Furthermore, there is a demand for mobility of vital sign monitoring devices. Vital sign devices are typically limited in their mobility by storage capacity and power generation capability. For example, a continuously monitored vital sign can quickly fill computer storage of a portable device and quickly consume battery power. Monitoring a person for dizziness or fainting continuously, including during inactive periods, e.g. sitting, unnecessarily consumes both storage and power.

Accelerometers which detect motion have been included with some vital sign devices, such as athletic fitness devices which monitor pulse rate and motion rate. However, such devices do not discriminate body motion, such as changing from a sitting position to a standing position, and consume power, to measure and track motion.

The following discloses a new and improved motion triggered vital sign measurement device and method which addresses the above referenced issues, and others.

In accordance with one aspect, a medical monitoring device includes a motion detection device and a vital sign monitoring device communicatively connected to the motion detection device. The motion detection device is adapted to detect whole body motion of a person. The vital sign monitoring device is adapted to monitor at least one vital sign for a predetermined time interval, in response to the detected whole body motion.

In accordance with another aspect, a method of monitoring vital signs includes detecting whole body motion of a person. At least one vital sign is monitored for a predetermined time interval in response to the detected whole body motion.

In accordance with another aspect, a medical monitoring device includes a motion detection device, a vital sign monitoring device, a vital sign evaluation device, and an alarm device. The motion detection device is adapted to detect body motion of a person indicative of a change in position, e.g. from sitting to standing. The vital sign monitoring device is communicatively connected to the motion detection device and adapted to monitor e.g. blood pressure for a predetermined time interval in response to the detected body motion. The vital sign evaluation device is adapted to evaluate the monitored blood pressure for conditions indicative of orthostatic intolerance. The alarm device is configured to generate a signal in response to the evaluated conditions

One advantage is ability to monitor persons for syncope in a non-hospital setting.

Another advantage resides in alarms for possible falls due to fainting, dizziness, or syncope.

Another advantage resides in reduced power and memory requirements.

Still further advantages will be appreciated to those of ordinary skill in the art upon reading and understanding the following detailed description.

The invention may take form in various components and arrangements of components, and in various steps and arrangement of steps. The drawings are only for purposes of illustrating the preferred embodiments and are not to be construed as limiting the invention.

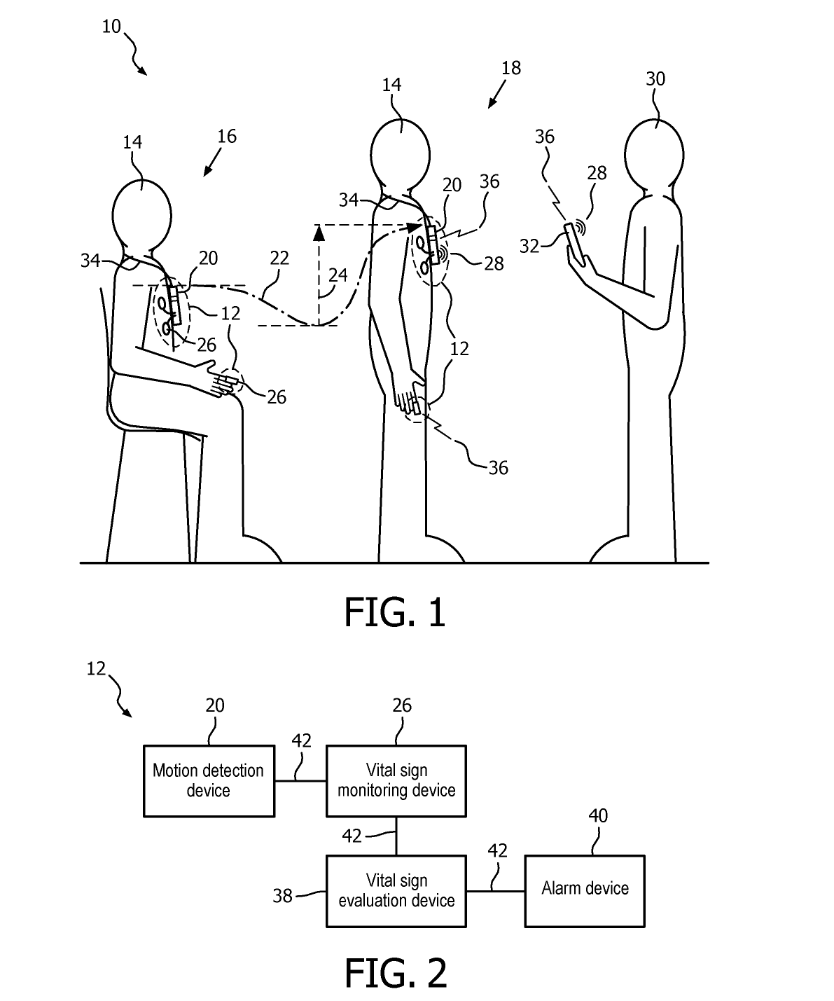

FIG. 1 diagrammatically illustrates an embodiment of the motion triggered vital sign measurement system in a time sequence.

FIG. 2 schematically illustrates one embodiment of the motion triggered medical monitoring device.

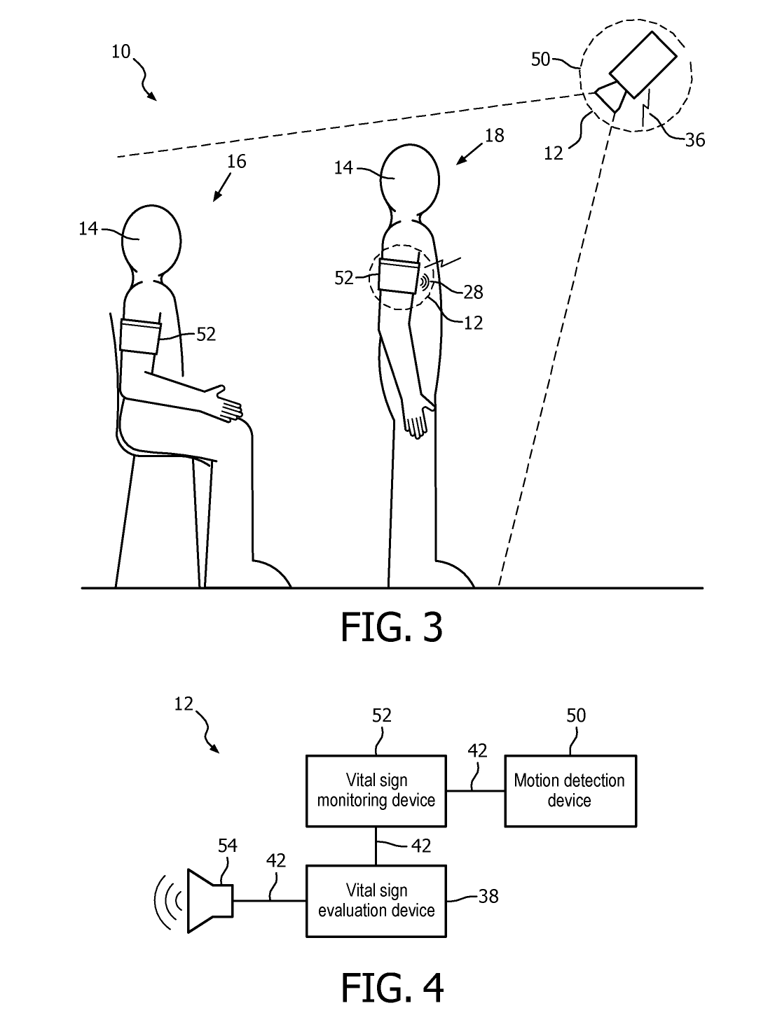

FIG. 3 diagrammatically illustrates another embodiment of the motion triggered vital sign measurement system in a time sequence.

FIG. 4 schematically illustrates another embodiment of the motion triggered medical monitoring device with an optical motion detection device.

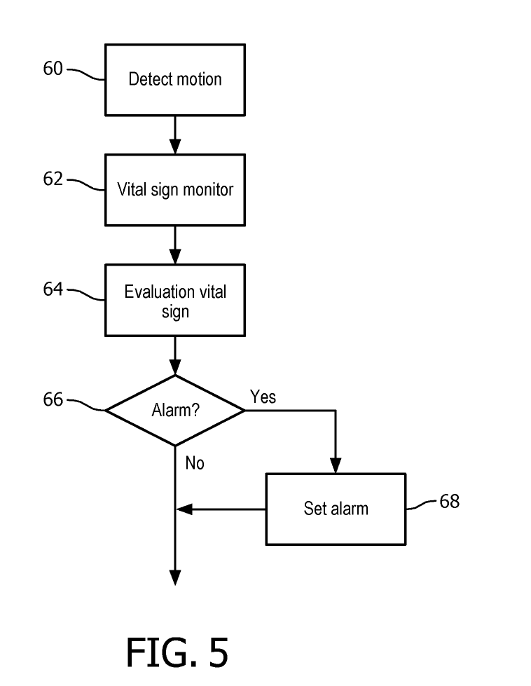

FIG. 5 flowcharts one method of motion triggered vital sign measurement.

With reference to FIG. 1, an embodiment of the motion triggered vital sign measurement system 10 in a time sequence is diagrammatically illustrated. The system includes a medical monitoring device 12, adapted to affix to a person 14 and monitor the person. The device 12 operates in two states, a resting state 16, and a motion triggered state 18. In the resting state 16, the device does not monitor patient vital signs, or if intermittent monitoring is required in resting state, the intermittent monitoring frequency is e.g. lower compared to the intermittent monitoring frequency in the motion triggered state. In the motion triggered state 18, the device actively monitors vital signs of the person for a predetermined interval triggered by detected whole body motion at a motion triggered state 18, either continuously or intermittent. For example, as a person moves from a sitting position to a standing position, orthostatic stress is applied to the person. The system is ideally suited for use as a fall prediction and prevention system.

A motion detection device or means 20 detects whole body motion. For example, as the person moves to a standing position 18, whole body movement 22, such as an change in elevation 24 relative to the center of gravity, is detected. The change in elevation creates orthostatic stress. The detected motion exceeds a predetermined threshold for whole body motion. For example, the person at rest generates motion, and the motion detection device discriminates between whole body motion indicative of standing and other motions such as respiration. In one embodiment, the motion detection device separates the acceleration vectors for elevation and other whole body movement. The motion detection device is located and/or affixed to the person in a position to discriminate whole body motion from fine motor motion. The motion detection device determines acceleration vectors that separate elevation changes from other body motion vectors. For example, as a person stands, the motion includes forward movement, not to be confused with a leaning movement, e.g. forward motion vector, or a twisting movement, e.g. left-right motion vector without standing. The motion detection device can alternatively detect motion with accelerometers, a multi-axis accelerometer, barometric pressure sensor, video monitoring, ultrasound monitoring, gyroscope, GPS receiver, or the like.

A vital sign monitoring device or means 26, is triggered by the detected whole body movement to monitor one or more vital signs, such as blood pressure, pulse, ECG, SpO₂ and the like. The vital sign monitoring device monitors the vital signs for a predetermined period of time, such as x minutes. For example, the person sits at a time t₀ and stands at a time t₁, and the vital sign monitoring device 26 monitors blood pressure, continuously or intermittently, in a cuff-less configuration for a time interval of 45 minutes beginning at t₁. The predetermined time interval can be adjusted according to medical guidelines in which syncope presents. In one embodiment, the intermittent monitoring includes an adjustable sampling rate. For example, the rate is adjusted to sample frequently in an initial period after standing, and steps down the sampling rate according to medical guidelines and/or patient population statistics after the initial period. Inactive monitoring during a resting state, and active monitoring limited to the predetermined interval, reduces power and memory or computer storage consumption. Blood pressure can also be detected with a cuff.

The medical monitoring device 12 evaluates the monitored vital sign, e.g. the signals generated by the vital sign monitoring device 24, such as blood pressure. The vital sign evaluation includes a determination of symptoms of syncope, such as a minimum blood pressure or a threshold change in blood pressure. For example, the blood pressure measured over time after the detected motion is compared to blood pressure versus time curves indicative or predictive of syncope. Such curves can be determined by data mining patient records of patient's that have suffered syncope for preceding blood pressure variations. As another option, the monitored blood pressure can be compared with a preselected low blood pressure value, e.g. indicative of low blood flow to the brain.

The medical monitoring device 12 generates an alert signal in response to a minimum blood pressure and/or threshold change in blood pressure. The medical monitoring device 12 can audibly sound and/or visually indicate an alert 28. A caregiver 30 responds to the alert to prevent the person experiencing symptoms of syncope, e.g. dizziness, fainting, etc., from falling. The alert can be sent to a computing device 32 of the caregiver. The alert can also alert the patient to sit or recline before becoming unconscious.

The device 12 includes a mechanism or means 34 to affix to the patient, such as a strap, clip, band, adhesive, and/or the like. For example, the motion detector device is affixed to the torso, or around the neck with a strap.

The device 12 can be configured with physically connected pieces with wired communication or physically separated pieces communicatively connected 36 via a wireless communication device, such as through a radio frequency (RF) transceiver or near body communication device. For example, the motion device communicates with an electrocardiogram (ECG) monitoring device and a photoplethysmogram (PPG) communication through skin contact and the near body communication. In another example, the pieces communicate using Bluetooth or 802.x protocols and RF communications.

With reference to FIG. 2 one embodiment of the motion triggered medical monitoring device 12 is schematically illustrated. The device 12 includes the motion detection device or means 20 to detect whole body motion indicative of orthostatic stress. For example, the motion detection device 20 includes an accelerometer, gyroscope, barometer, GPS receiver and/or the like to detect body motion and a processor or other software device which analyzes the body motion to determine whether whole body motion has occurred. The analysis can be based on changes in elevation, relative vertical motion vectors, horizontal motion vectors, and the like.

In response to the detected body motion being determined to be whole body motion, e.g. a vertical motion vector exceeding the threshold, a signal is communicated to the vital sign monitoring device 26. The vital sign monitoring device, in one embodiment, includes a cuff-less blood pressure (BP) measuring device such as two pulse sensing devices, e.g. ECG, PPG, and the like, which are used to determine the pulse wave velocity between the pulse sensing devices. The blood pressure is proportional to the pulse wave velocity. The cuff-less BP measuring device uses the signals of the ECG and PPG to measure or determine BP, e.g. systolic/diastolic blood pressure. The measures can be continuous or repeated measures over the time interval.

A vital sign evaluation device or means 38 is communicatively connected to the vital sign monitoring device 26, receives the monitored vital signs, and evaluates the received vital signs for symptoms of dizziness, fainting, and/or syncope which can cause the person to fall. For example, with the systolic blood pressure less than 90 mm Hg, or the diastolic blood pressure less than 60 mm Hg, the vital sign evaluation device generates an alarm signal in response. In another example, with a change in systolic or diastolic greater than 20 mm Hg, the vital sign evaluation device generates an alarm signal in response. The vital sign evaluation device can also compare other physiological values, such as heart rate or SpO₂, to thresholds. In another embodiment, time evolution patterns of physiological parameters are compared with patterns indicative of syncope or other potential events.

The vital sign evaluation device is communicatively connected to one or more alarm devices 40. The alarm device includes an audible and/or visual indicator, such as sounded alarm and/or flashing light. The alarm device includes the computing device of the caregiver, which can indicate the alarm, the person's identity, and/or location. For example, as the person stands, the motion detection device 20 detects an elevated whole body motion of 20 cm which triggers monitoring blood pressure by the vital sign monitoring device for a 45 minute period. During the 45-minute period, the vital sign evaluation device evaluates the monitored blood pressure, determines whether blood pressure drops more than 20 mm Hg and, in response to the evaluated drop in blood pressure, generates the alarm signal. The alarm signal received by the alarm device sets the audible indicator and flashing light attached to the medical monitoring device 12, indicating to the caregiver that the person is in danger of falling. Simultaneously, the alarm signal is communicated to the portable computing device 32, which records the alarm signal, and/or indicates with a flashing message display and sound that the person is in danger of falling.

The motion detection device 20, vital sign monitoring device 26, vital sign evaluation device 38, and alarm device 40 are communicatively connected with a communication device or means 42. The communication device 42 includes a wired connection in a wired configuration with devices physically connected. The communication device 42 includes a wireless connection in a wireless configuration with devices physically separated. The wireless connection includes RF, infrared, or near body communication. The medical monitoring device can include different configurations between component devices. For example, the motion detection device can be in the wired configuration with the ECG device and in a wireless configuration with the PPG device.

With reference to FIG. 3, another embodiment of the motion triggered vital sign measurement system 10 in a time sequence is diagrammatically illustrated. The person 14 moves from the resting state 16 at time t₀ to standing 18 at time t₁. The motion detection device includes an optical motion detector 50, such as one or more cameras and a processor configured to view body motion of the person, e.g. identify and track movement indicative of orthostatic stress. For example, the camera receives images of the person in the field of view of the camera moving from the sitting position to the standing position. The processor communicatively connected to receive the images uses object recognition to identify the person and analyze the motion. For example, initial objects can include facial recognition, a badge or device worn by the patient, and the like, and then a growing algorithm to identify and associate other body parts. Alternatively, the person wears an energy emitter, such as an LED, ultrasound emitter, infrared LED, RF transmitter, or the like. The emitted energy is received by appropriate stationary mounted receivers which use triangulation to monitor motion and distance and duration moved.

The medical monitoring device 12, in communication with the optical motion detector, receives a signal indicating whole body motion. The medical monitoring device 12, in one embodiment, includes a cuff-based vital sign monitoring device 52 adapted to monitor blood pressure of the person. The medical monitoring device 12 includes the components integrated as a single physical device adapted to fit over the upper arm. For example, the medical monitoring device includes a pressure cuff to measure blood pressure, and the pressure cuff slides over the upper arm of the person and is adjustable, to be held in place by an attachment mechanism, such as elastic band, hook and loop, etc. The vital sign evaluation device and alarm device are physically attached to the pressure cuff. The cuff can include a region of a characteristic color to assist in discriminating between monitored persons and bystanders.

With reference to FIG. 4, another embodiment of the medical monitoring device 12 with an optical motion detection device 50 or means is schematically illustrated. The optical motion detection device 50, such as the camera and configured or programmed processor, views the person at time t₀ person in the sitting position, and at time t₁ person moving to standing position. The optical motion detection device 50 uses object recognition with motion separation to determine a change in position, e.g. a change in altitude of a center of mass, a characteristic point, or the like, giving rise to orthostatic stress. The motion detection device signals the vital sign monitoring device 52, e.g. pressure cuff measuring blood pressure for the predetermine time interval. The vital sign evaluation device 38 in response to blood pressure indicative of a minimum blood pressure, or a threshold change in blood pressure, signals the alarm device 54, which includes a speaker that sounds the audible signal.

In one embodiment, the system is a person worn fall prevention system which alerts the person or caregiver or relative of a potential fall before the fall occurs. In another embodiment, the system is a fall detection system that signals for help before or when a fall occurs.

The various devices 20, 26, 38, 40, 50, 52 include or share one or more electronic data processing devices, such as a processor or electronic processing device powered by either solar and/or battery power. The optical motion detection device 50 can include a AC/DC power source. Moreover, the disclosed vital sign evaluation and communication techniques are suitably implemented using a non-transitory storage medium storing instructions (e.g., software) readable by an electronic data processing device and executable by the electronic data processing device to perform the disclosed techniques.

With reference to FIG. 5, one method of motion triggered vital sign measurement is flowcharted. In a step or by a module 60, body motion of a person is detected, visually and/or physically. The motion is analyzed to determine whether the motion is whole body motion indicative of orthostatic stress, such as a change in elevation by moving from the sitting to the standing position.

At least one vital sign is monitored by the vital sign monitoring device for a predetermined time interval in response to the detected whole body motion in a step or by a module 62. For example, in response to the person standing, the blood pressure of the person is monitored using a cuff-less 26 or pressure cuff 42 blood pressure monitor.

In a step or by a module 64, the at least one measured monitored vital sign is evaluated for orthostatic stress. For example, blood pressure is monitored for a minimum blood pressure, or a threshold change in blood pressure.

The alarm is signaled in response to an evaluated measure in a decision step or by a module 66. For example, in response to monitored blood pressure dropping below a minimum value or changing more than a threshold value, the alarm condition is signaled.

The alarm is sent in response to the alarm signal in a step or by a module 68. For example, the alarm signal causes an audible sound, tone, noise, or voice message indicative of the person in danger of falling. In another example, the alarm signal sets both the audible indicator and a visual indicator such as a light signal or a message on a caregiver display.

The communicating of conditions between modules or devices includes the detected motion, the monitored vital signs, the evaluated measures, and the alarm signal. The communicating can include devices physically distributed or separated. The devices or modules can include attachment mechanisms which attached the device or component to the person. A non-transitory computer-readable storage medium carrying software which controls one or more electronic data processing devices to perform the steps of communicating, detecting and/or analyzing motion, monitoring vital signs, evaluating vital signs, and signaling an alarm.

It is to be appreciated that in connection with the particular illustrative embodiments presented herein certain structural and/or functional features are described as being incorporated in defined elements and/or components. However, it is contemplated that these features may, to the same or similar benefit, also likewise be incorporated in other elements and/or components where appropriate. It is also to be appreciated that different aspects of the exemplary embodiments may be selectively employed as appropriate to achieve other alternate embodiments suited for desired applications, the other alternate embodiments thereby realizing the respective advantages of the aspects incorporated therein.

It is also to be appreciated that particular elements or components described herein may have their functionality suitably implemented via hardware, software, firmware or a combination thereof. Additionally, it is to be appreciated that certain elements described herein as incorporated together may, under suitable circumstances, be stand-alone elements or otherwise divided. Similarly, a plurality of particular functions described as being carried out by one particular element may be carried out by a plurality of distinct elements acting independently to carry out individual functions, or certain individual functions may be split up and carried out by a plurality of distinct elements acting in concert. Alternately, some elements or components otherwise described and/or shown herein as distinct from one another may be physically or functionally combined where appropriate.

In short, the present specification has been set forth with reference to preferred embodiments. Obviously, modifications and alterations will occur to others upon reading and understanding the present specification. It is intended that the invention be construed as including all such modifications and alterations insofar as they come within the scope of the appended claims or the equivalents thereof. That is to say, it will be appreciated that various of the above-disclosed and other features and functions, or alternatives thereof, may be desirably combined into many other different systems or applications, and also that various presently unforeseen or unanticipated alternatives, modifications, variations or improvements therein may be subsequently made by those skilled in the art, which are similarly intended to be encompassed by the following claims.

1. A medical monitoring device, comprising:
a motion detection device adapted to detect whole body motion of a person; and
a vital sign monitoring device communicatively connected to the motion detection device and adapted to monitor at least one vital sign for a predetermined time interval, either continuously or intermittently, in response to the detected whole body motion;
wherein the vital sign monitoring device is configured to measure blood pressure of a person, and includes either a cuff-less or a cuff-based blood pressure measuring device.

2. The device according to claim 1, wherein the detected whole body motion includes a determined change in elevation.

3. The device according to claim 1, wherein the determined change in elevation corresponds to a position of the person changing from a sitting position to a standing position.

4. The device according to claim 1, wherein the motion detection device includes at least one of:
an optical monitoring device adapted to monitor movements of the person;
an ultrasound monitoring device adapted to monitor movements of the person;
an accelerometer;
a gyroscope;
a barometer; or
a GPS receiver.

5. (canceled)

6. The device according to claim 1, wherein the cuff-less blood pressure measuring device includes a plurality of pulse sensing devices configured to measure pulse wave velocity.

7. The device according to claim 1, further including:
a vital sign evaluation device adapted to evaluate measures of the at least one monitored vital sign for orthostatic intolerance.

8. The device according to claim 7, wherein the evaluated measures include at least one of:
a minimum systolic or diastolic blood pressure; or
a threshold reduction in systolic or diastolic blood pressure.

9. The device according to claim 7, wherein the vital sign evaluation device is further configured to signal an alarm in response to an evaluated measure.

10. The device according to claim 1, further including:
a communications device adapted to communicate at least one of:
a prediction that a fall is imminent;
the detected motion;
the monitored at least one vital sign;
the evaluated measures; or
the alarm signal.

11. A method of monitoring vital signs, comprising:
detecting whole body motion of a person; and
monitoring at least one vital sign for a predetermined time interval in response to the detected whole body motion; and
measuring blood pressure of the person with either a cuff-less or a cuff-based blood pressure measuring device.

12. The method according to claim 11, wherein detecting whole body motion includes determining a change in elevation.

13. (canceled)

14. The method according to claim 11, further including:
evaluating measures of the at least one monitored vital sign for orthostatic stress; and
signaling an alarm in response to an evaluated measure.

15. The method according to claim 11, further including:
communicating at least one of:
a prediction that a fall is imminent;
the detected motion;
the monitored at least one vital sign;
the evaluated measures; or
the alarm signal.

16-20. (canceled)

21. The device according to claim 7, further including:
an alarm device configured to generate a signal in response to the evaluated conditions.

22. The device according to claim 22, further including:
an attachment mechanism adapted to attach the motion detection device to the person to at least one of: around the neck, to the torso, or upper arm, the signal predicting or indicating the person falling.
